(19) United States
(12) Patent Application Publication
(10) Pub. No.: US 2010/0040632 A1
(43) Pub. Date: Feb. 18, 2010

(54) COMPOSITION

(75) Inventor: Mala K. Maini, London (GB)

Correspondence Address: BANNER & WITCOFF, LTD., 1100 13th STREET, N.W., SUITE 1200, WASHINGTON, DC, 20005-4051, US

(73) Assignee: UCL Business PLC, London (GB)

(21) Appl. No.: 12/530,559

(22) PCT Filed: Mar. 7, 2008
(86) PCT No.: PCT/GB08/00811

§ 371 Date: Oct. 23, 2009

(30) Foreign Application Priority Data
Mar. 9, 2007 (GB) 0704651.9

(51) Int. Cl. A61K 39/395; C12N 5/00; C12N 15/63; A61K 38/00; A61K 31/7088

(52) U.S. Cl. 424/158.1; 435/325; 435/320.1; 514/12; 514/44 R

(57) ABSTRACT

The present invention relates to a method of treating liver damage, especially hepatitis B virus (HBV) related liver damage. The present invention also relates to compositions and kits for use in treating liver damage.

The present invention relates to a method of treating liver damage, especially hepatitis B virus (HBV) related liver damage. The present invention also relates to compositions and kits for use in treating liver damage.

Chronic infection with Hepatitis B Virus (HBV), a hepatotropic DNA virus, is a major cause of liver disease worldwide. More than 350 million people are persistently infected and at risk of developing chronic liver inflammation resulting in liver cirrhosis and hepatocellular carcinoma. The World Health Organisation estimates that at least 1 million deaths each year are directly attributable to HBV-related liver disease. An increasing proportion of the chronic disease seen in many countries is due to the development of viral variants lacking the expression of ‘e’ antigen but associated with active viral replication and liver disease, termed ‘e’ antigen negative chronic hepatitis B (eAg-CHB) (1). Patients with eAg-CHB are prone to recurrent, spontaneous ‘hepatic flares’, characterised by large, unexplained fluctuations in liver inflammation and a propensity to progress rapidly to severe liver fibrosis (2). These hepatic flares provide a window of opportunity to study mechanisms involved in dynamic alterations in viral load and liver inflammation over a condensed timeframe. Since HBV is non-cytopathic, liver damage is thought to be immune-mediated, but the molecular pathways leading to hepatocyte death in human HBV infection are not well understood. There is a pressing need to dissect the ways in which different components of the immune response contribute to liver disease in HBV infection as this will allow the rational development of immunotherapeutic strategies that enhance viral control whilst limiting or blocking liver inflammation.

Immune-mediated liver damage in patients with HBV infection has conventionally been attributed to cytolytic killing of infected hepatocytes by virus-specific CD8 T cells. However, this assumption was challenged by previous work, demonstrating the presence of activated HBV-specific CD8 T cells at high frequencies in the livers of patients controlling HBV infection without any evidence of liver inflammation. Instead, the distinguishing feature between patients with or without HBV-related chronic liver disease was the presence of a large, non-antigen-specific lymphocytic infiltrate in the livers of the former group (3). The mechanisms resulting in the recruitment and activation of this non-specific inflammatory infiltrate have been explored in the transgenic mouse model of HBV. In this model it was possible to reduce the severity of liver damage by inhibiting the non-specific cellular infiltrate (4, 5), reinforcing the concept that liver inflammation initiated by virus-specific CD8 is amplified by other lymphocytes (6).

One of the largest constituents of the lymphocytic infiltrate in HBV transgenic mice is NK cells (NK1.1+CD3−), with a 10-12 fold increase in their numbers in the inflammatory infiltrate compared to baseline (4, 5). NK cells (CD3⁻ CD56⁺) are likewise a major component of the cellular infiltrate in the human liver, comprising 30-40% of total intrahepatic lymphocytes (7). An early rise in circulating NK cells has been documented in the incubation phase of HBV infection, suggesting they may contribute to the initial viral containment in this setting (8). The antiviral and pathogenic potential of NK cells in patients with chronic HBV infection has not previously been addressed.

The mechanism through which NK cells mediate anti-viral cytotoxicity appears to be organ dependent (9). NK cytotoxicity through perforin/granzyme is now considered to be of less relevance in the liver environment, where the target hepatocytes are relatively resistant to lysis through this pathway (9, 10). Receptor-mediated cell death through ligand/receptor pairs belonging to the TNF superfamily is likely to play a more important role in liver damage (11, 12). One such pathway is mediated through TNF-related apoptosis-inducing ligand (TRAIL) (13) expressed on infiltrating lymphocytes interacting with TRAIL death-inducing receptors (TRAIL-R1, TRAIL-R2) (14) on hepatocytes. This has been shown to be a critical mechanism of liver damage in vivo in Listeria and concanavalin-A-induced hepatitis in mice (15). An essential role for NK cells in hepatic TRAIL-mediated apoptosis was highlighted in the setting of the surveillance of tumour metastases (16). Normal human hepatocytes have also been shown to be sensitive to TRAIL-mediated apoptosis (17, 18), and it has been suggested that susceptibility to this pathway may be increased during viral hepatitis (19, 20). The inventors have hypothesised that NK expressed TRAIL may play a role in non-antigen-specific mediation of liver damage in chronic HBV infection.

In Liang et al., (48), the use of a soluble TRAIL receptor to block TRAIL function was shown to reduce hepatitis and hepatic cell death in HBV transgenic mice. In Liu et al. (56), human soluble death receptor 5 was used to block TRAIL function and shown to reduce apoptosis of HBV-transfected hepatocytes.

NK cell effector function is a result of the balance of signals through their activatory and inhibitory receptors, a balance that is influenced by the local cytokine milieu. Furthermore, NK cells can be directly activated to anti-viral activity by certain cytokines, with IFN-α promoting cytotoxicity (21) and TRAIL expression (22), and IL-12 favouring IFN-γ production (21). IFN-α production characterises the early stages of acute viral infections but it is unclear whether its release can also be triggered by fluctuations in viral load occurring on the background of the persistent high level antigenic stimulation found in chronic HBV infection. The downstream effects of any IFN-α produced may be attenuated in antigen-activated cells (23, 24) or modified by an increase in other cytokines such as IL-1β (25) and IL-8 (26, 27).

There is a need for an effective treatment of liver damage, especially liver damage caused by a HBV infection. There is also a need for an effective treatment of hepatic flares associated with HBV infection. There is also a need to overcome the detrimental effects of IFN-α, namely hepatocyte cell death, especially in individuals with advanced liver disease.

According to a first aspect the present invention there is provided the use of an IL-8 blocking agent in the manufacture of a medicament for the treatment and/or prophylaxis of liver disease.

It has surprisingly been found that IL-8 blocking agents are effective in treating and preventing liver disease in the absence of a TRAIL blocking agent.

In a specific embodiment of the first aspect of the present invention the medicament does not comprise a TRAIL blocking agent.

In an alternative specific embodiment of the first aspect of the present invention the medicament does additionally comprise a TRAIL blocking agent.

It has been found that the use of a TRAIL blocking agent and an IL-8 blocking agent is particularly effective at treating and preventing liver disease. In particular, the combination of both blocking agents has been found to be more effective than the use of a TRAIL blocking agent alone.

The term “IL-8 blocking agent” as used herein refers to any agent capable of blocking the activity of IL-8, especially the activity of IL-8 to induce the expression of TRAIL-R2 on hepatocytes and/or the activity of IL-8 to chemoattract NK cells. Suitable blocking agents include soluble IL-8 receptors, antibody molecules having affinity for IL-8 or its receptor, other molecules having affinity for IL-8 or its receptor (e.g. Affibodies), small molecules, etc.

The term “antibody molecule” as used herein refers to polyclonal or monoclonal antibodies of any isotype, or antigen binding fragments thereof, such as Fv, Fab, F(ab′)₂ fragments and single chain Fv fragments. The antibody molecule may be a recombinant antibody molecule, such as a chimeric antibody molecule, a CDR grafted antibody molecule or an antigen binding fragment thereof. Such antibodies and methods for their production are well known in the art. The antibody molecule can be produced in any suitable manner, e.g. using hybridomas or phage technology. One skilled in the art would know how to produce an antibody having the desired affinity, see Antibodies: A Laboratory Manual, eds. Harlow et al. Cold Spring Harbour Laboratory 1988. The antibody molecule can be produced from any suitable organism, for example, from sheep, mice, rats, rabbits, goats, donkeys, camels, lamas or sharks or from a library of specificities generated through molecular biology techniques.

It is particularly preferred that the IL-8 blocking agent is an antibody molecule having affinity for IL-8 or its receptor. A number of antibodies having affinity for IL-8 are known (e.g. ABX-IL-8 (Mahler et al., Chest, 126, 926-34, 2004)).

The term “TRAIL blocking agent” as used herein refers to any agent capable of preventing TRAIL mediated apoptosis. Preferably the blocking agent prevents the interaction between the TRAIL ligand and the TRAIL death inducing receptors (TRAIL-R1 and TRAIL-R2). Suitable blocking agents include soluble TRAIL receptors (e.g. soluble death receptor (see Liu et al., (56) and Liang et al., (48)), antibody molecules having affinity for the TRAIL ligand or receptor, other molecules having affinity for the TRAIL ligand or receptor (e.g. Affibodies), small molecules, etc.

It is particularly preferred that the TRAIL blocking agent is an antibody molecule having affinity for the TRAIL ligand or receptor.

A number of antibodies having affinity for the TRAIL ligand are known (e.g. TRAIL-PE (Pharmingen BD Biosciences, Cowley, UK)), as well as antibodies having affinity for the TRAIL death inducing receptors (e.g. mAb 375, TRAIL-R1-PE and TRAIL-R2-PE (R&D Systems, Abingdon, UK))

The use of an IL-8 blocking agent alone or the use of a combination of a TRAIL blocking agent and an IL-8 blocking agent have been found to be particularly effective at treating and/or preventing liver disease.

In a particularly preferred embodiment of the first aspect of the present invention there is provided the use of a TRAIL blocking agent and an IL-8 blocking agent in the manufacture of a medicament for the treatment and/or prophylaxis of liver disease.

The term “liver disease” as used herein refers to any liver disease wherein the TRAIL pathway is leading to the apoptosis of hepatocytes. The liver disease may be associated with HBV or HCV infections, co-infections of HBV or HCV with HIV, or may be fatty acid liver disease. Preferably the liver disease is associated with HBV infection. It is further preferred that the liver disease is a chronic HBV infection, preferably an eAg-CHB infection. It is still further preferred that the liver disease involves hepatic flares characterised by large increases in liver inflammation, and is associated with chronic HBV infections.

In the use according to the first aspect of the present invention, the medicament may additionally comprise IFN-α. IFN-α is often used to treat viral infections as it promotes NK cells to become cytotoxic. Accordingly, the inventors consider that IFN-a activated NK cells have a dual role in viral control and liver damage. Furthermore, the exogenous use of IFN-α in the treatment of HBV infections is often limited by its tendency to cause a hepatic flare and hence liver damage, especially in individuals with advanced liver disease. Accordingly, by administering IFN-α with an IL-8 blocking agent and/or a TRAIL blocking agent, the detrimental effects of IFN-α (i.e., liver damage) can be reduced. Any form of IFN-α can be used provided it functions as an antiviral agent. Preferably the IFN-α is PEGylated.

In a further use according to the first aspect of the present invention, the medicament may additionally comprise a reverse transcriptase antiviral. Reverse transcriptase antivirals are often used to treat viral infections and their use is associated with hepatic flares. The exogenous use of reverse transcriptase antivirals in the treatment of HBV infections is often limited by loss of viral suppression resulting from the development of drug resistance, leading to a hepatic flare and hence liver damage, especially in individuals with advanced liver disease. Accordingly, an IL-8 blocking agent and/or a TRAIL blocking agent may be used to prevent hepatic flares forming due to the loss of viral suppression resulting from the development of drug resistance to reverse transcriptase antivirals. Furthermore, by administering a reverse transcriptase antiviral with an IL-8 blocking agent and/or a TRAIL blocking agent, the detrimental effects of the reverse transcriptase antiviral (i.e., liver damage) can be reduced. Any reverse transcriptase antiviral can be used that is for treating HBV infection, including lamivudine, adefovir, entecavir, clevudine, tenofovir and combinations of these.

In the use according to the first aspect of the present invention, the medicament may be used to treat liver disease in individuals who are receiving IFN-α or a reverse transcriptase antiviral. As the use of IFN-α and reverse transcriptase antivirals have a tendency to be associated with the development of hepatic flares, the medicament will be particularly useful in preventing or reducing the hepatic flares in individuals receiving IFN-α or reverse transcriptase antivirals.

In the use according to the first aspect of the present invention, the medicament can also comprise any additional component that assists with the treatment and/or prophylaxis of liver disease. Suitable additional components include anti-viral agents when the liver disease is associated with a viral infection (e.g. a HBV infection or a HBV and HIV co-infection). Suitable anti-HBV and anti-HIV agents include nucleoside inhibitors.

Each component of the medicament can be delivered simultaneously, sequentially or separately to an animal capable of raising an immune response. Preferably, each component is given simultaneously. The composition can be given repeatedly.

In the use according to the first aspect of the present invention, the medicament is for treatment of liver disease in any suitable animal, such as a human, livestock or pets. Preferably the animal is a mammal or a bird. In particular, the animal may be selected from the group comprising: human, dog, cat, cow, horse, pig, sheep and birds. It is specifically preferred that the animal is a human.

The term “treatment” as used herein refers to any reduction in a measure of liver inflammation and damage, such as serum transaminases, or a reduction in liver inflammation on biopsy.

The term “prophylaxis” as used herein refers to preventing, delaying or attenuating the level of liver inflammation and damage.

As will be appreciated by those skilled in the art, the IL-8 blocking agent and/or the TRAIL blocking agent can be administered in the form of one or more nucleic acids encoding the blocking agent or agents. Accordingly, in an alternative embodiment of the first aspect of the present invention, there is provided the use of one or more nucleic acid molecules encoding an IL-8 blocking agent in the manufacture of a medicament for the treatment and/or prophylaxis of a liver disease. Preferably the medicament also comprises one or more nucleic acids encoding a TRAIL blocking agent.

The use of one or more nucleic acids to deliver the blocking agent or agents to the desired site is an alternative method for treating liver disease. When both the TRAIL blocking agent and the IL-8 blocking agent are encoded on one or more nucleic acids, they can be encoded on a single nucleic acid molecule or on separate nucleic acid molecules.

The one or more nucleic acids of the present invention can be obtained by methods well known in the art. For example, naturally occurring sequences may be obtained by genomic cloning or cDNA cloning from suitable cell lines or from DNA or cDNA derived directly from the tissues of an organism such as a human or mouse. Alternatively, the sequences can be synthesized using standard synthesis methods such as the phosphoramidite method.

Numerous techniques may be used to alter the nucleic acid sequence obtained by the synthesis or cloning procedures. Such techniques are well known to those skilled in the art. For example, site directed mutagenesis, or oligonucleotide directed mutagenesis and PCR techniques may be used to alter the DNA sequence. Such techniques are well known to those skilled in the art and are described in a vast body of literature known to those skilled in the art.

The one or more nucleic acid molecules are preferably expression vectors. Expression vectors are well known for expressing nucleic acids in a variety of different organisms, including mammalian cells. Preferably the expression vector of the present invention comprises a promoter and an operably linked nucleic acid molecule encoding one or more of the blocking agents. It is further preferred that the vector comprises any other regulatory sequences required to obtain expression of the nucleic acid molecule.

Suitable regulatory sequences include sequences that will ensure that the nucleic acid sequence is expressed in the desired location within the body, i.e., the liver.

The present invention provides an expression vector encoding a TRAIL blocking agent and an IL-8 blocking agent.

The present invention also provides a host cell transformed with one or more nucleic acid molecules encoding the TRAIL blocking agent and the IL-8 blocking agent. The blocking agents can be encoded on a single nucleic acid molecule or on separate nucleic acid molecules.

The term “transformation” refers to the insertion of an exogenous nucleic acid molecule into a host cell, irrespective of the method used for insertion, for example direct uptake, transduction, f-mating or electroporation. The exogenous nucleic acid may be obtained as a non-integrating vector (episome), or may be integrated into the host's genome.

Preferably the host cell is a eukaryotic cell, more preferably a mammalian cell, such as Chinese hamster ovary (CHO) cells, HPMCs, HeLa cells, baby hamster kidney (BHK) cells, cells of hepatic origin such as HepG2 cells, and myeloma or hybridoma cell lines. Preferably the host cell is of hepatic origin.

According to a second aspect, the present invention provides a method for the treatment and/or prophylaxis of an individual with liver disease comprising delivering an effective amount of an IL-8 blocking agent to the individual.

In a specific embodiment of the second aspect of the present invention the method does not comprise delivering TRAIL blocking agent to the individual.

In an alternative specific embodiment of the second aspect of the present invention the method additionally comprises delivering an effective amount of a TRAIL blocking agent to the individual.

In a particularly preferred embodiment of the second aspect of the present invention there is provided a method for the treatment and/or prophylaxis of an individual with liver disease comprising delivering an effective amount of a TRAIL blocking agent and an IL-8 blocking agent to the individual.

The method according to the second aspect of the present invention may additionally comprise delivering an effective amount of IFN-α to the individual. An effective amount of IFN-α is an amount that suppresses HBV replication.

The method according to the second aspect of the present invention may also additionally comprise delivering an effective amount of a reverse transcriptase antiviral to the individual. An effective amount of the reverse transcriptase antiviral is an amount that suppresses HBV replication.

In the method according to the second aspect of the present invention, any additional component that assists with the treatment and/or prophylaxis of liver disease can also be delivered to the individual. Suitable additional components are described above with reference to the first aspect of the present invention.

In the method according to the second aspect of the present invention may be used to treat liver disease in individuals who are receiving IFN-α or a reverse transcriptase antiviral. As IFN-α and reverse transcriptase antivirals have a tendency to result in hepatic flares, the method will be particularly useful in preventing or reducing the hepatic flares in individuals receiving IFN-α or a reverse transcriptase antiviral.

As indicated above with respect to the first aspect of the present invention, the IL-8 blocking agent and/or the TRAIL blocking agent can be administered in the form of one or more nucleic acids encoding the blocking agent or agents. Accordingly, in an alternative embodiment of the second aspect of the present invention, there is provided a method for the treatment and/or prophylaxis of an individual with liver disease comprising delivering an effective amount of one or more nucleic acid molecules encoding an IL-8 blocking agent to the individual. Preferably the method additionally comprises delivering an effective amount of one or more nucleic acids encoding a TRAIL blocking agent to the individual.

When both the TRAIL blocking agent and the IL-8 blocking agent are encoded on one or more nucleic acids, they can be encoded on a single nucleic acid molecule or on separate nucleic acid molecules.

According to a third aspect of the present invention there is provided the use of a TRAIL blocking agent in the manufacture of a medicament for the treatment and/or prophylaxis of hepatic flares.

It has surprisingly been found that TRAIL blocking agents are effective in reducing and/or preventing hepatic flares.

Hepatic flares are acute episodes of increased inflammation of the liver. Such hepatic flares can be associated with alcohol abuse or viral infections. The term “hepatic flares” preferably refers to hepatic flares caused by a HBV infection or a HBV and HIV co-infection. The HBV infection is preferably an eAg-CHB infection. Preferably the hepatic flares are caused by an eAg-CHB infection.

In the use according to the third aspect of the present invention the medicament may additionally comprise one or more of an IL-8 blocking agent, IFN-α or a reverse transcriptase antiviral. The presence of such additional agents can improve the treatment and/or prophylaxis of hepatic flares.

In the use according to the third aspect of the present invention, the medicament can also comprise any additional component that assists with the treatment and/or prophylaxis of hepatic flares. Suitable additional components are described above with respect to the first aspect of the present invention.

In the use according to the third aspect of the present invention, the medicament may be used to treat hepatic flares in individuals who are receiving IFN-α or a reverse transcriptase antiviral. As IFN-α and reverse transcriptase antivirals have a tendency to result in hepatic flares, the medicament will be particularly useful in preventing or reducing the hepatic flares in individuals receiving IFN-α or a reverse transcriptase antiviral.

Those skilled in the art will appreciate that the TRAIL blocking agent can be administered in the form of one or more nucleic acids encoding the TRAIL blocking agent. Accordingly, in an alternative embodiment of the third aspect of the present invention, there is provided the use of one or more nucleic acid molecules encoding a TRAIL blocking agent in the manufacture of a medicament for treating hepatic flares.

According to a fourth aspect, the present invention provides a method for the treatment and/or prophylaxis of an individual with hepatic flares comprising delivering an effective amount of a TRAIL blocking agent to the individual.

The method may additionally comprise delivering an effective amount of one or more of an IL-8 blocking agent, IFN-α or a reverse transcriptase antiviral to the individual.

In the method according to the fourth aspect of the present invention, any additional component that assists with the treatment and/or prophylaxis of hepatic flares can be delivered to the individual. Suitable additional components are described above with respect to the first aspect of the present invention.

The method according to the fourth aspect of the present invention may be used to treat hepatic flares in individuals who are receiving IFN-α or a reverse transcriptase antiviral. As IFN-α and reverse transcriptase antivirals have a tendency to cause hepatic flares, the method will be particularly useful in preventing or reducing the hepatic flares in individuals receiving IFN-α or a reverse transcriptase antiviral.

Those skilled in the art will appreciate that the TRAIL blocking agent can be administered in the form of one or more nucleic acids encoding the TRAIL blocking agent. Accordingly, in an alternative embodiment of the fourth aspect of the present invention, there is provided a method for the treatment and/or prophylaxis of an individual with hepatic flares comprising delivering an effective amount of one or more nucleic acid molecules encoding a TRAIL blocking agent to the individual.

The present invention also provides a pharmaceutically acceptable composition comprising a TRAIL blocking agent and an IL-8 blocking agent, or one or more nucleic acids encoding a TRAIL blocking agent and an IL-8 blocking agent, together with one or more pharmaceutically acceptable excipients. The composition may additionally comprise IFN-α or a reverse transcriptase antiviral, or a nucleic acid molecule encoding IFN-α or a reverse transcriptase antiviral.

The present invention also provides a pharmaceutically acceptable composition comprising a TRAIL blocking agent in combination with IFN-α or a reverse transcriptase antiviral, or one or more nucleic acid molecules encoding a TRAIL blocking agent and IFN-α or a reverse transcriptase antiviral, together with one or more pharmaceutically acceptable excipients.

The present invention also provides a pharmaceutically acceptable composition comprising a IL-8 blocking agent in combination with IFN-α or a reverse transcriptase antiviral, or one or more nucleic acid molecules encoding a IL-8 blocking agent and IFN-α or a reverse transcriptase antiviral, together with one or more pharmaceutically acceptable excipients.

Suitable excipients are well known to those skilled in the art.

The specific amounts of each component of the pharmaceutically acceptable compositions of the present invention can be determined using standard methodologies and by extrapolating from the specific values used in the example section below. The specific amounts used will depend on a number of factors, including the size and metabolism of the animal to be treated.

The pharmaceutical compositions of the present invention may be administered in any suitable manner, including orally, parenterally or via an implanted reservoir. Preferably the pharmaceutical composition is administered orally or by injection.

The pharmaceutical composition may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical composition of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The present invention also provides any one of the pharmaceutical compositions of the present invention for use in therapy, especially treatment of a liver disease.

The present invention also provides a kit for treating a liver disease comprising a TRAIL blocking agent and an IL-8 blocking agent, or one or more nucleic acids encoding a TRAIL blocking agent and an IL-8 blocking agent. The kit may additionally comprise IFN-α or a reverse transcriptase antiviral, or a nucleic acid molecule encoding IFN-α or a reverse transcriptase antiviral.

The present invention also provides a kit for treating hepatic flares comprising a TRAIL blocking agent in combination with IFN-α or a reverse transcriptase antiviral, or one or more nucleic acid molecules encoding a TRAIL blocking agent and IFN-α or a reverse transcriptase antiviral.

The present invention also provides a kit for treating a liver disease comprising a IL-8 blocking agent in combination with IFN-α or a reverse transcriptase antiviral, or one or more nucleic acid molecules encoding a IL-8 blocking agent and IFN-α or a reverse transcriptase antiviral.

The present invention is now described by way of example only with reference to the following figures.

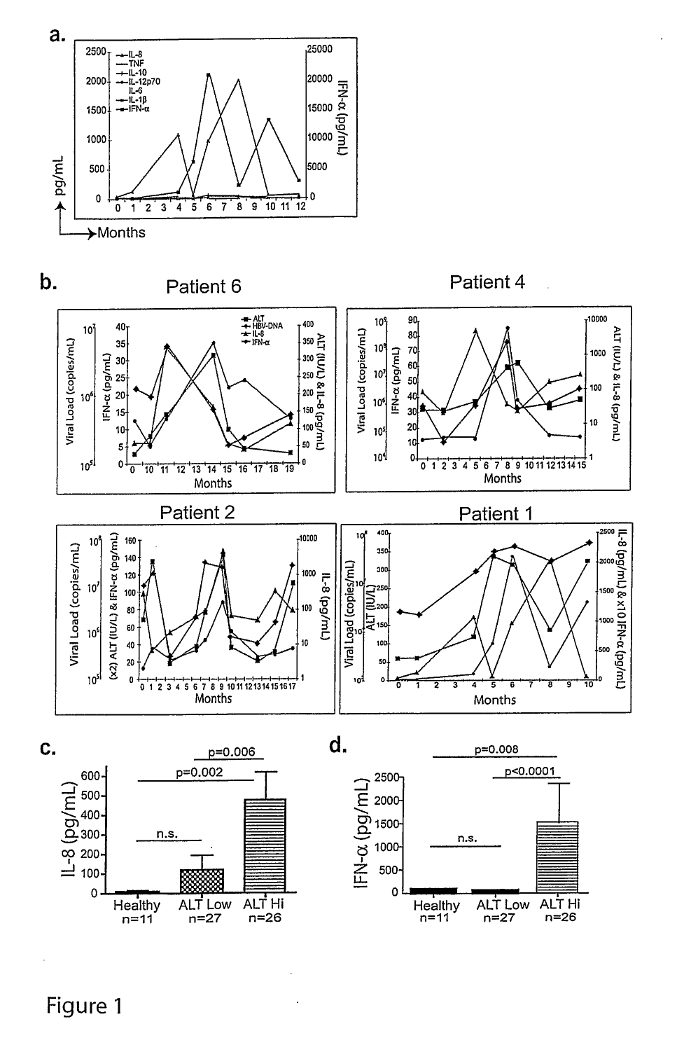

FIG. 1 shows that IL-8 and IFN-α concentrations are elevated in the serum of CHB patients with liver inflammation. (a) Circulating concentrations of multiple cytokines detected in longitudinal serum samples taken from a representative patient (patient 1), assayed by CBA (IL-8, IL-1β, IL-6, IL-10, TNF, IL-12p70) and sandwich ELISA (IFN-α). The concentrations of inflammatory cytokines were determined by CBA software or Prism. (b) Temporal relationship between serum IL-8 and IFN-α concentrations and liver inflammation (ALT) and viral load (HBV-DNA) in 4 representative patients of 14 patients assayed. Cross-sectional comparison of IL-8 (c) and IFN-α (d) levels quantitated by sandwich ELISA in healthy donors, HBV patients with low ALT (ALT<60 IU/l for the last year), and HBV patients with raised ALT (ALT>60 IU/L at time of sampling). Significance testing was done using the Mann Whitney U test.

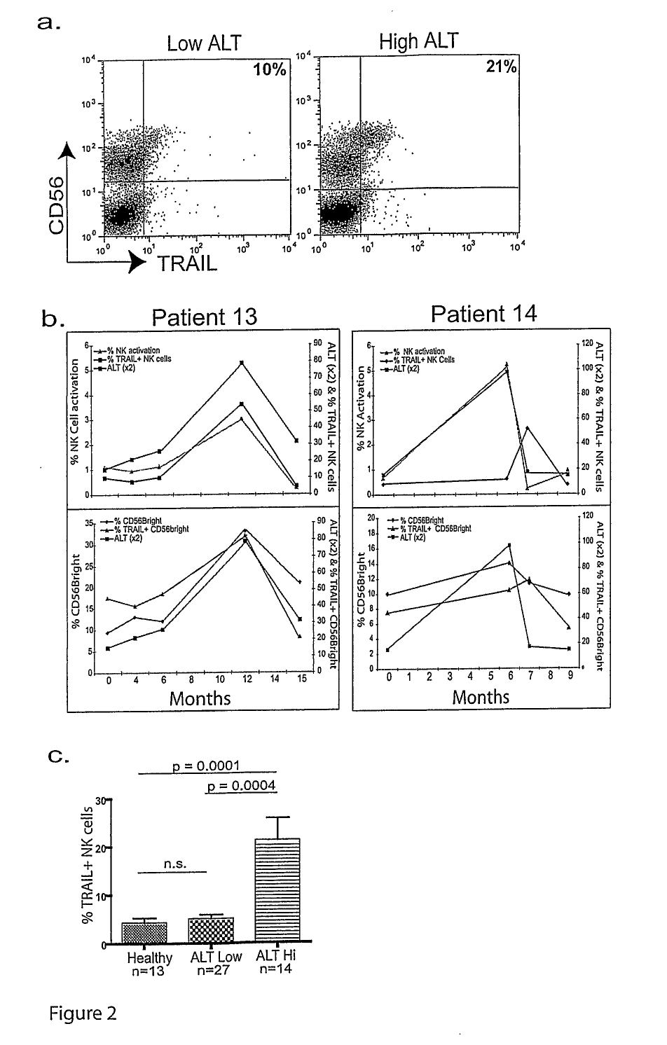

FIG. 2 shows direct ex vivo correlation between NK cell TRAIL expression and liver inflammation in CHB patients. (a) Representative flow cytometry dot plot from a CHB patient stained with mAb to CD3, CD56 and TRAIL, and gated on CD3⁻ cells. The percentages denote the proportion of freshly isolated CD3⁻CD56⁺ NK cells staining with TRAIL. (b) Upper panels: PBMC from patients with eAg-CHB were stained ex vivo and the percentage of NK (CD3⁻CD56⁺) expressing TRAIL upon flow cytometry was correlated with ALT. CD69+ NK cells are presented as a percent of total lymphocytes. Lower panels: The percent of CD56^{bright} NK cells out of total CD3⁻ CD56⁺ NK cells and the percent of those CD56^{bright} NK cells that were TRAIL positive was plotted against ALT. (c) Cross sectional analysis of ex vivo surface TRAIL expression on CD3⁻CD56⁺ NK cells from healthy donors, HBV patients with low ALT (ALT<60 IU/l for the last year), and HBV patients with raised ALT (ALT>60 IU/L at time of sampling). Significance testing was done using the Mann Whitney U test.

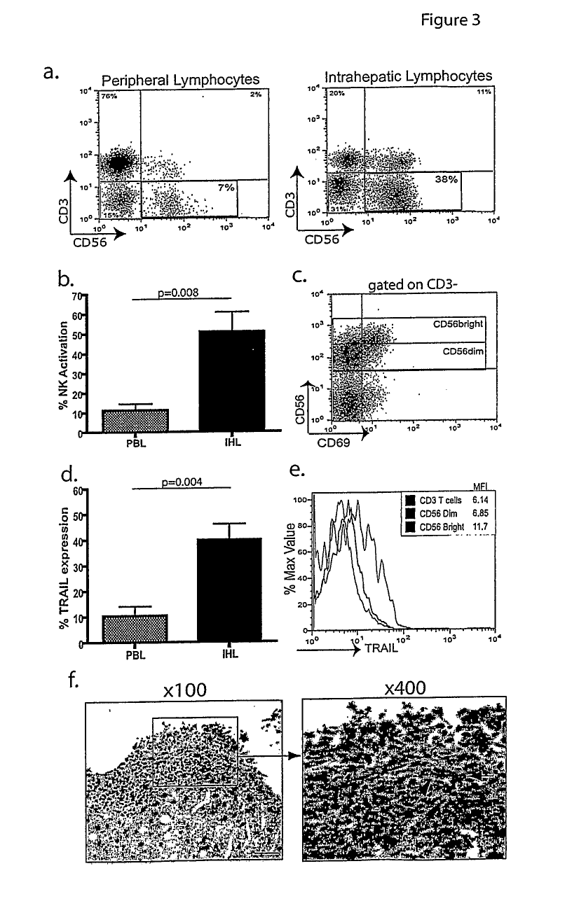

FIG. 3 shows enrichment of NK cell numbers, TRAIL expression and activation in the liver compared to periphery. (a) Mononuclear cells from the periphery and liver of a representative CHB patient were stained with antibodies to CD3 and CD56, and the proportion of CD3⁺ T cells, CD3⁺CD56⁺ NKT cells and CD3⁻CD56⁺ NK cells (highlighted in box) determined by flow cytometry. NK cells (CD3⁻CD56⁺) from liver-infiltrating (IHL) and circulating (PBL) lymphocytes from five chronically infected HBV patients were assessed ex vivo for CD69 expression (b) and TRAIL expression (d). P values were determined by the Mann-Whitney U test. (c) Flow cytometry dot plot analysis of a representative CHB patient comparing intra-hepatic NK cell activation in the CD56^{bright} and CD56^{dim} NK cell subsets. (e) A histogram comparing TRAIL expression on the CD56^{brigh} and CD56^{dim} NK cell subsets and CD3⁺ T cells isolated from the liver. (f) Paraffin-embedded liver sections taken from seven HBV patients were stained with an anti-TRAIL mAb. The boxed area on the left panel indicates the field of view on the right panel. TRAIL positive cells are stained brown and are highlighted with black arrows.

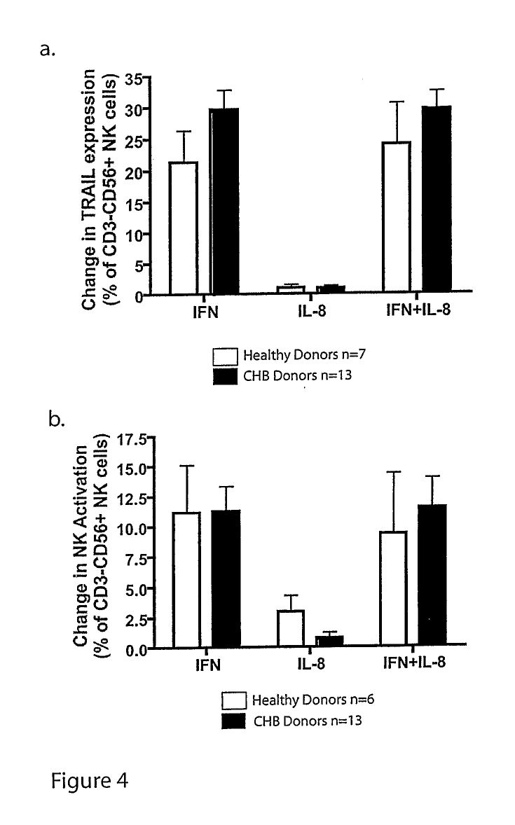

FIG. 4 shows that concentrations of IFN-α observed in patient sera induce increased surface TRAIL expression and activation of NK cells isolated from CHB patients. PBMC from healthy donors (white bars) and CHB patients (black bars) were incubated for 24 h in vitro with IFN-α (1000 U/mt), IL-8 (5 ng/mL) or IFN-α and IL-8. The effect of this incubation on TRAIL expression (a) and NK cell activation (b) was assessed by flow cytometry analysis with NK cells identified as CD3⁻CD56⁺. Graphs were plotted by subtracting baseline levels of CD69 or TRAIL observed in the untreated controls from those observed after cytokine treatment.

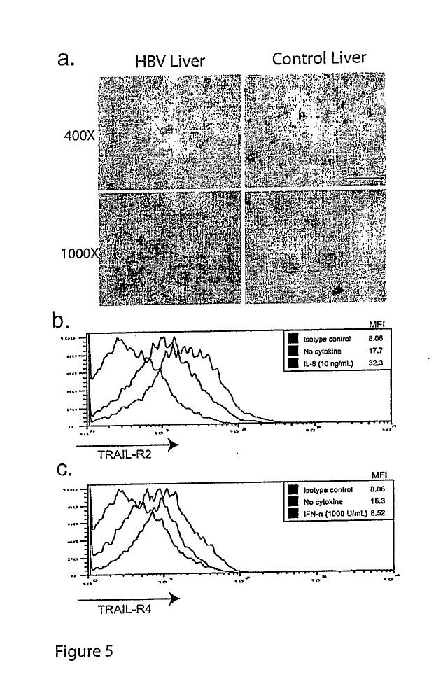

FIG. 5 shows TRAIL receptor expression on hepatocytes in HBV infection. (a) Paraffin-embedded sections from HBV-infected (left panels) and healthy control (right panels) livers were stained with an anti-TRAIL-R2 mAb. Membrane localised (arrows) and cytoplasmic (*) TRAIL-R2 expression is indicated by the brown chromogen reactivity. b) MFI of HepG2 TRAIL-R2 levels after IL-8 incubation (10 ng/mL for 24 h) compared to untreated and isotype controls. (c) MFI of HepG2 TRAIL-R4 levels after IFN-α (1000 U/mL) for 24 h compared to untreated and isotype controls. These are representative of 5 separate experiments.

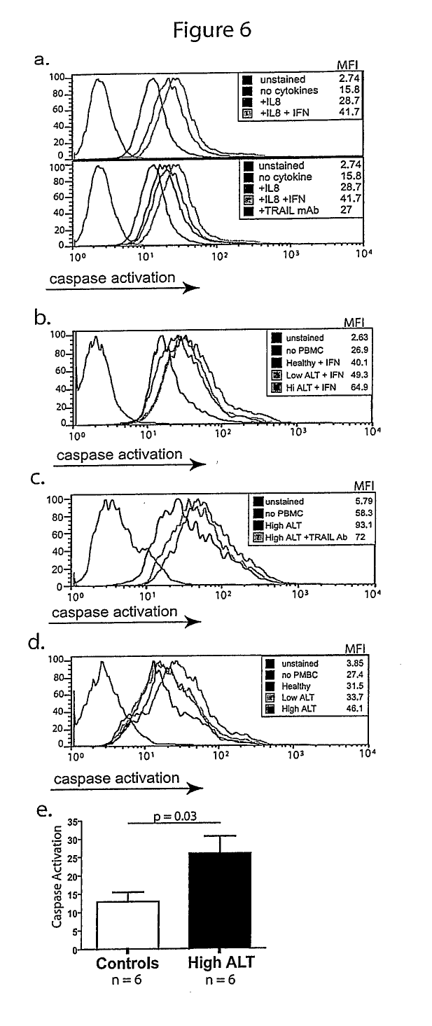

FIG. 6 shows that IFN-α: activated NK cells from CHB patients can mediate TRAIL-induced hepatocyte apoptosis. (a) HepG2 cells were incubated for 24 h with or without IL-8 (10 ng/mL). Simultaneously, PBMC were incubated for 24 h with or without IFN-α (1000 U/mL). Upper panel: PBMC were then added to HepG2 at a E:T ratio of 10:1 for 4 h before visualisation of caspase activation with the fluoroscein labelled Z-VAD-fink and detection by flow cytometry, expressed as mean fluorescence intensity (MFI). Lower panel: Experimental procedure as above except for addition of a TRAIL blocking antibody (10 ng/mL). (b) Representative results of PBMC from healthy donors, CHB patients with low ALT and CHB patients with high ALT incubated with IFN-α (1000 U/mL) for 24 h and then assessed for caspase activation of IL-8-treated HepG2 as above. (c) Representative HepG2 caspase induction by ex vivo PBMC from high ALT HBV patient and reduction upon addition of TRAIL blocking mab. (d) Representative HepG2 caspase induction upon addition of PBMC taken directly ex vivo from a healthy donor, CHB patient with low ALT and CHB patient with high ALT. (e) Summary level of HepG2 caspase induction using PBMC directly ex vivo from HBV patients with liver injury (ALT high patients, n=6) compared to PBMC from controls (HBV patients without raised ALT n=3 and healthy controls n=3) (p=0.03, Mann Whitney U test).

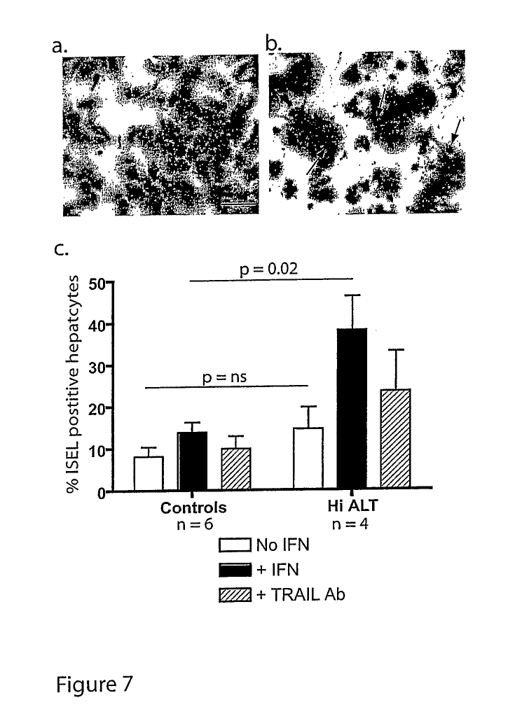

FIG. 7 shows that NK cells from CHB patients can mediate TRAIL induced apoptosis in primary human hepatocytes. Primary human hepatocytes were cultured for 48 h with addition of IL-8 (10 ng/mL) and IFN-α (1000 U/mL) for the last 24 h. Concurrently, PBMC from three healthy donors, three CHB patients with low ALT and four CHB patients with high ALT were incubated with or without IFN-α for 24 h at 37° C. The hepatocytes and PBMC were incubated together for 18 h at an E:T ratio of 10:1, with or without a TRAIL blocking antibody in the interferon-treated wells. The degree of apoptosis was determined by in situ DNA end labelling (ISEL) for the detection of DNA fragmentation. (a) A representative image of control hepatocytes incubated without PBMC. (b) A representative image of hepatocytes after 18 h incubation with PBMC from a CHB patient with high ALT. The arrows represent ISEL positive hepatocytes. (c) Summary data of % ISEL-positive hepatocytes using PBMC from high ALT patients (n=4) versus controls (low ALT patients n=3 and healthy donors n=3) without (white bars) or with (black bars) IFN-α treatment and with TRAIL-blocking of IFN-α treated wells (hatched bars). Results are presented after subtraction of the mean baseline level of hepatocyte apoptosis of 14% seen without addition of PBMC and significance tested with the Mann Whitney U test.

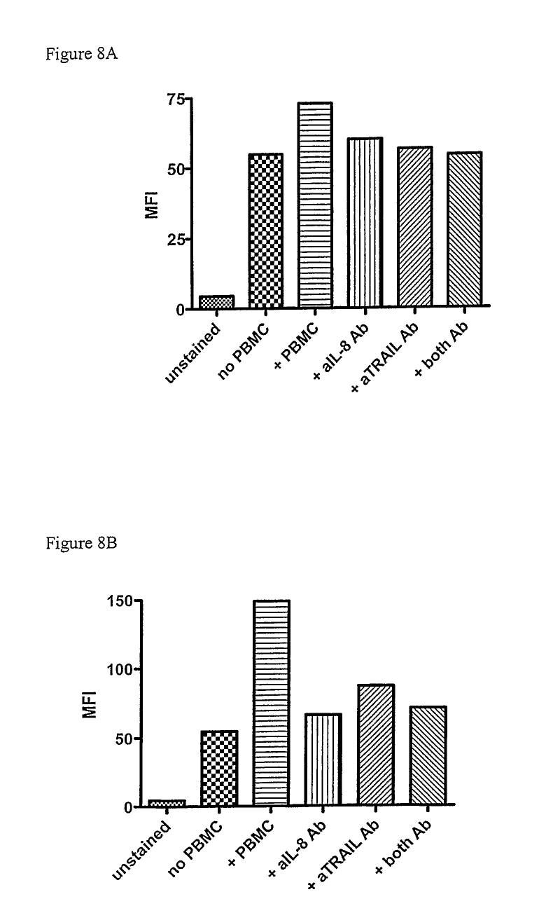

FIG. 8 shows the result of using an anti-IL-8 antibody in combination with an anti-TRAIL antibody to prevent apoptosis of hepatocytes HepG2 cells were incubated for 24 h without IL-8. Simultaneously, PBMC from two different donors ((a) NB84; (b) GE83)) were incubated for 24 h with IFN-α (1000 U/mL). PBMC were then added to HepG2 at a E:T ratio of 10:1 for 4 h in the presence or absence of 1 μg/ml of an anti-IL-8 monoclonal antibody and/or a anti-TRAIL monoclonal antibody (10 ng/mL) before visualisation of caspase activation with the fluoroscein labelled Z-VAD-fmk and detection by flow cytometry, expressed as mean fluorescence intensity (MFI).

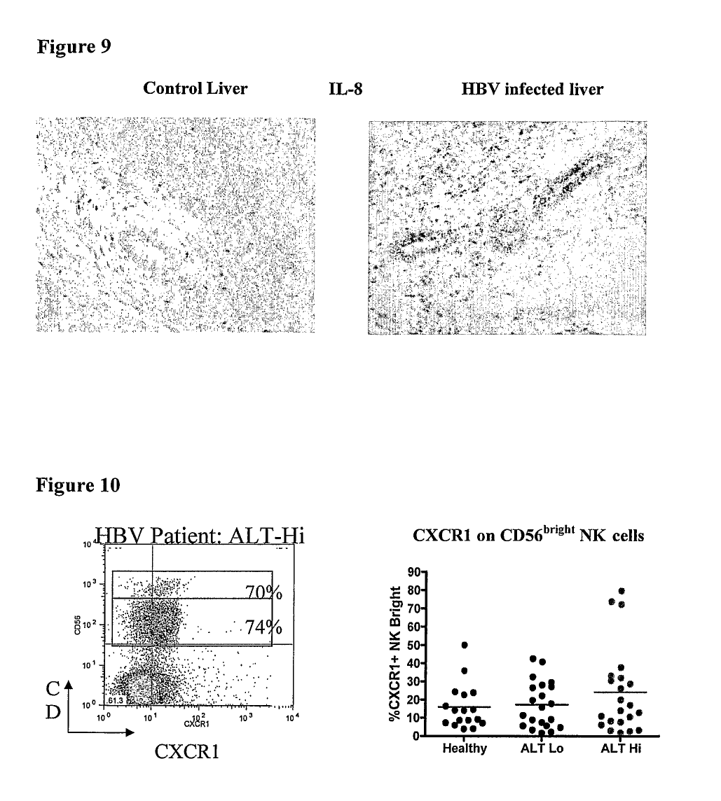

FIG. 9 shows increased IL-8 staining of hepatocytes from livers of patients that have been infected by HBV compared to hepatocytes from livers without HBV infection.

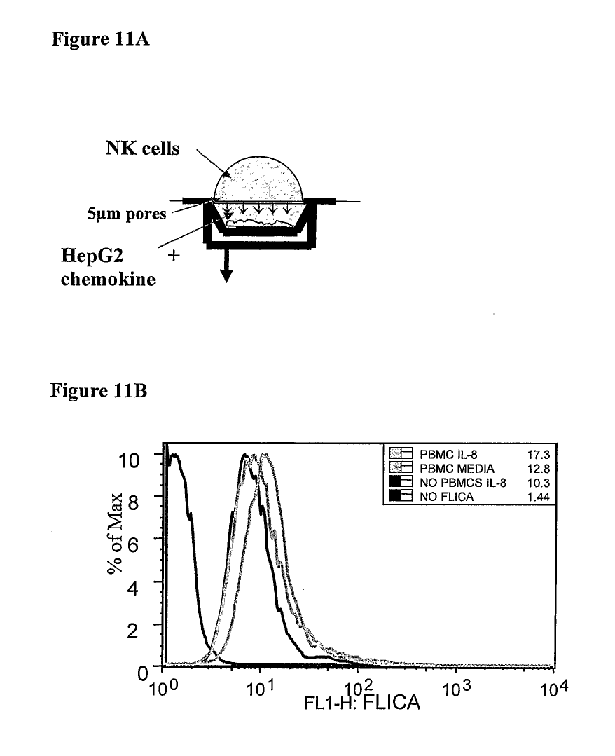

FIG. 10 shows that the high affinity IL-8 receptor (CXCR1) is expressed by NK cells in HBV infected individuals including the CD56^{bright} subset of NK cells.

FIG. 11 shows the results of a functional chemotaxis assay. (A) shows the assay set-up. (B) shows the amount of apoptosis induction of hepatocytes by NK cells chemoattracted by IL-8 (versus that induced by NK cells migrating without addition of chemokine).

### EXAMPLES

In order to understand the cytokine milieu influencing NK cell activity, the inventors quantified IFN-α and a number of other key pro-inflammatory and immunoregulatory cytokines in patients with chronic HBV infection. The inventors took advantage of a cohort of well-characterised patients with eAg-CHB sampled repeatedly before, during and after multiple hepatic flares to correlate sensitive measurements of their serum cytokine levels with changes in liver inflammation. The inventors observed large fluctuations in serum IFN-α and IL-8 concentrations in association with the hepatic flares. Increases in circulating IFN-α and IL-8 in CHB patients with liver inflammation were accompanied by an increase in NK cell activation and surface TRAIL expression measured directly ex vivo. The inventors then explored the mechanisms underlying these ex vivo observations, which could explain the resultant liver damage. The inventors established that the concentrations of IFN-α and IL-8 produced in vivo promoted the TRAIL pathway of NK cell killing, acting on both the ligand and the receptors. The inventors confirmed that, in the presence of this combination of cytokines, NK cells from patients with chronic HBV infection became capable of TRAIL-mediated killing of hepatocytes.

### Materials and Methods

### Patients and Controls

Seventy two patients with chronic HBV infection (HBsAg positive) were recruited with full ethics approval and informed consent, with 11 patients being HBeAg positive and the remainder HBeAg negative and anti-HBeAb positive (measured by commercial enzyme immunoassay kits, Murex Diagnostics, Dartford, UK). HBV-DNA viral load was quantified by the Roche Amplicor Monitor Assay (Roche Laboratories). The patients were negative for antibodies to Hepatitis C Virus and Hepatitis Delta Virus, and to HIV-1 and 2 (Ortho Diagnostic System, Murex Diagnostics). None of the patients included in the study were taking antiviral therapy or immunosuppressive drugs. Sera were obtained and immediately frozen from 53 patients, PBMC from 46 patients and liver biopsies/explants or paraffin-embedded sections from twenty patients. A subset of fourteen HBeAg negative CHB patients was subjected to longitudinal analysis, with multiple serum and PBMC samples taken (Table 2). Serum samples were analysed in parallel, and PBMC analysed directly ex vivo.

Control samples consisted of sera and PBMC from 14 and 13 healthy donors respectively and paraffin-embedded liver sections from 4 healthy donors and 4 patients with alcoholic hepatitis.

### Antibodies and Reagents

The antibodies CD3⁻Cy5.5/PerCP, CD56-FITC, TRAIL-PE, CD69-APC (Pharmingen, BD Biosciences, Cowley, UK), TRAIL-R1-PE, TRAIL-R2-PE, TRAIL-R3-PE, TRAIL-R4-PE, anti-IL8 and CD56-PE (R&D Systems, Abingdon, UK) were used for flow cytometric analyses at manufacturers recommended concentrations unless stated otherwise. The anti-TRAIL antibody for neutralisation of bioactivity (R&D Systems) was used at a concentration of 10 ng/mL. Recombinant human IFN-α2a (rhIFN-α; PBL Biomedical Laboratories, Piscataway, N.J., USA) and recombinant human IL-8 (rhIL-8; R&D Systems) were used at concentrations stated for each experiment.

### Determination of Serum Cytokine Concentrations

Serum cytokine concentrations were ascertained using the Cytometric Bead Array (CBA) Inflammation kit (BD Biosciences) to manufacturers protocols. Briefly, 50 μL of patient serum or standard recombinant protein dilutions was added to a mixture of capture beads coated with mAb to a panel of cytokines (IL-8, IL-1β, IL-6, IL-10, TNF, IL-12p70) and a PE-conjugated detection reagent. After 3 hours, the capture beads were washed and acquired on FACSCaliber flow cytometer (BD Biosciences). Using the recombinant standards and the BD CBA Software provided, cytokine concentrations were quantified for each serum sample. Serum IFN-α was assayed using a standard sandwich ELISA kit (PBL Biomedical Laboratories) where 50 μL of patient serum was analysed according to manufacturers High Sensitivity protocol.

### Ex Vivo Staining of NK Cells

Freshly isolated PBMC from HBV patients and healthy donors, or intrahepatic lymphocytes isolated from HBV patients as described previously (3) were incubated for 30 minutes at 4° C. with antibodies to CD3, CD56, CD69 and TRAIL. PBMC were washed twice with PBS+ 1% FCS and fixed with 1% para-formaldehyde before acquisition on a FACSCaliber flow cytometer. Isotype-matched control mAbs were used for defining positive populations staining with the CD69 and TRAIL-specific mAbs.

### Immunohistochemistry of Liver Samples for TRAIL and TRAIL Receptors

Archival paraffin blocks from 15 CHB, 4 alcoholic liver disease cases and 4 healthy donors were stained for the expression of TRIAL receptor 1 and 2. Serial sections from 7 eAg-CHB patients were stained for expression of TRAIL. Sections (4 μm) were cut onto charged slides (Surgipath, UK) and heated for 1 h at 60° C. After deparaffinising and rehydration, sections were treated in 0.3% H₂O₂ in water to block endogenous peroxidase activity. Antigen retrieval was performed using the ALTER technique as previously described (54). Following a brief wash in water, sections were placed onto a Sequenza (Shandon, UK) and washed in TBS/Tween pH 7.6. Monoclonal antibodies to TRAIL-R1 (1:100 dilution, R&D Systems) or TRAIL-R2 (1:50 dilution, R&D Systems) were applied for 40 minutes at room temperature. Sections were washed in TBS/Tween and antibody detected using Dako Chemate Envision horseradish peroxidase kit (Dako, UK). Sections were washed in water, counterstained in heamatoxylin, dehydrated, placed into xylene and mounted in DPX.

### Cytokine Induced NK Cell Activation and Upregulation of TRAIL Expression

PBMC were resuspended in supplemented RPMI 10% FCS, plated into a round bottom 96 well tissue culture plate at 3×10⁵ cells/well and incubated with rhIFN-α (1000 U/mL), rhIL-8 (5 ng/mL) or IFN-α & IL-8 for 24 hours at 37° C. The degree of cytokine induced NK activation and upregulated TRAIL expression was determined by subtracting baseline CD69 or TRAIL expression from that observed after cytokine treatment.

### Cytokine Induced Changes in Trail-R Expression on the HepG2 Hepatoma Cell Line

HepG2 hepatoma cells were trypsinised from a 75 cm² flask and plated into a 48 well flat bottom tissue culture plate at 2×10⁵ cells/well. The cells were allowed to adhere for 5 hours before the addition of rhIL-8 (10 ng/mL) or rhIFN-α (1000 U/mL) and incubated for 24 hours at 37° C. The wells were washed twice with PBS and then incubated on ice for 45 minutes with 5 mM EDTA. This gentle detachment from the plate prevented the loss of surface TRAIL-R expression. The cells were then washed twice with PBS+1% FCS to remove the EDTA before incubation for 30 minutes at 4° C. with mAbs to the four membrane bound TRAIL-R and acquisition on a FACSCaliber flow cytometer.

### NK Expressed TRAIL-Mediated Apoptosis of HepG2 Cell Line

HepG2 were trypsinised from a 75 cm³ flask, plated into a 48 well flat bottom tissue culture plate at 1×10⁵ cells/well and allowed to adhere. Adhered cells were incubated with and without IL-8 (10 ng/mL) or IFN-α (1000 U/mL) at 37° C. for 24 h. PBMC (or purified NK cells or NK-depleted PBMC) from chronic HBV patients were also incubated with and without IFN-α (1000 U/mL) at 37° C. for 24 h. After this incubation a TRAIL blocking antibody and/or a IL-8 blocking antibody was added to the relevant wells for 1 hour prior to the addition of PBMC to HepG2 wells at a ratio of 10:1 (PBMC:HepG2). After 4 h, the degree of caspase activation was determined using the Carboxyfluoroscein-FLICA apoptosis detection kit (Serotec, Kidlington, Oxford, U.K.) using the manufacturers protocol for detection by flow cytometry.

### NK Expressed TRAIL Mediated Apoptosis of Primary Human Hepatocytes.

Primary human hepatocytes were isolated from non-diseased liver explant tissue using collagenase perfusion (55), resuspended in Williams E medium containing hydrocortisone, insulin, glutamine, plated into 48 well flat bottom culture plate at 1×10⁵ cells per well and allowed to adhere for 2 h. Medium was replaced and cells rested for 24 h before stimulation for 24 h at 37° C. with IL-8 (10 ng/mL) and IFN-α (1000 U/mL). PBMC from CHB or healthy donors were incubated with or without IFN-α (1000 U/mL) at 37° C. for 24 h. After this time, a TRAIL blocking antibody (10 ng/mL) was added to the relevant well for 2 h before PBMC were added to hepatocytes at a ratio of 10:1 (PBMC:hepatocyte) and incubated for a further 18 h at 37° C. before fixing with methanol. The degree of apoptosis was determined by in situ DNA end labelling (ISEL) for the detection of DNA fragmentation (55). Briefly, the fixed cells were incubated with ISEL mixture (TBS pH 7.6 plus 5 mM MgCl, 10 mM 2-mercaptoethanol, 5 mg/mL bovine serum albumin, 20 units Klenow DNA polymerase (Bioline, London, U.K.), 0.01M of nucleotides dATP, dCTP & dGTP (Invitrogen, Paisley, U.K.), and digoxygenin labelled dUTP (Roche Laboratories)) for 1 hour at 37° C. The sections were then washed with distilled water and incubated with sheep anti-digoxygenin alkaline phosphatase conjugated Fab fragment (1:200 dilution; Roche Laboratories) for 1 hour at room temperature. After further washing in TBS pH 7.6 sections were incubated with alkaline phophatase substrate for 15 min, counterstained with Mayers haematoxylin and re-fixed in methanol at 4° C. Induction of apoptosis was quantified by an independent observer blinded to the study design, who counted at least 200 hepatocytes in each well.

### Results

### Large Fluctuations in Circulating Levels of IFN-α and IL-8 During Flares of Liver Disease in Chronic HBV Infection

Patients with eAg negative chronic hepatitis B (eAg-CHB) are susceptible to spontaneous ‘flares’ of liver inflammation associated with rapid changes in viral load. These flares provide an opportunity to investigate mechanisms of HBV-related liver damage during periods of dynamic fluctuation that are predictable enough to be captured upon longitudinal sampling. A cohort of fourteen eAg-CHB patients that had previously been identified as likely to undergo recurrent hepatic flares (2) were recruited and studied longitudinally. Through frequent sampling, serum was obtained before, during and after one or multiple flares, defined in this study as an abrupt increase in serum alanine transaminase (ALT) to more than double the baseline value and more than three times the upper limit of normal (<35 IU/L for women, <50 IU/L for men). Serum ALT was used as a surrogate marker for liver damage since studies in chimpanzees (28) and humans (29) infected with IIBV have shown that it accurately predicts histological findings of hepatic inflammation. All patients included had a well-characterised disease course with clinical monitoring for at least one year and between 4 and 10 serial samples available for the study (Table 2), usually taken at intervals of 1-2 months. Using the Inflammatory Cytometric Bead Array (CBA) kit and Enzyme Linked Immunosorbent Assay (ELISA) technology it was possible to quantitate multiple cytokines simultaneously from a small volume of serum. The cytokines analysed were interleukin-8 (IL-8), IL-1β, IL-6, IL-10, tumour necrosis factor (TNF), IL-12p70 and interferon-alpha (IFN-α). Of the seven cytokines examined, only IL-8 and IFN-α were consistently detected, with peak concentrations far in excess of those observed for the other five cytokines in patients, and significantly higher than in healthy controls (Table 1). The serum levels of these two cytokines were observed to undergo large fluctuations, which were recurrent in the cases with multiple flares (FIG. 1a), with patients consistently displaying substantial fold changes throughout the flaring events assayed (Table 2). These uniform, large fluctuations were not observed with the other cytokines measured (FIG. 1a).

The patients in this cohort had a marked degree of liver inflammation (indicated as maximum ALT, Table 2) and high viral load (see maximum viral load, Table 2) at the height of the flare. Changes in serum IFN-α and IL-8 levels showed a temporal association with fluctuations in ALT and HBV-DNA (FIG. 1b). For the majority of patients (10/14), the peak serum level of IL-8 preceded the onset of the flare of liver inflammation (the sample just prior to the ALT peak), either simultaneous to or immediately after a sharp increase in viral load (FIG. 1b and Table 2). Maximal serum concentrations of IFN-α occurred concurrently with the peak of liver inflammation (FIG. 1b & Table 2, median interval between IFN-α peak and ALT peak=0), at a time when IL-8 levels were declining but were still highly elevated compared to healthy controls (FIG. 1a & FIG. 1b).

To establish whether elevated levels of IL-8 and IFN-α were restricted to HBV patients with active disease or could also be found in the absence of liver inflammation, the inventors conducted a large cross-sectional study. Serum IFN-α and IL-8 concentrations were compared in controls without HBV infection, patients with chronic HBV infection with no evidence of liver inflammation (ALT<60 IU/L at the time of sampling and no ALT>60 IU/L recorded in the preceding year), and patients with HBV infection with liver inflammation (ALT>60 IU/L at the time of sampling). HBV patients with liver inflammation had significantly raised levels of both IL-8 (FIG. 1c) and IFN-α (FIG. 1d) compared to the control groups. In contrast, healthy donors consistently had low or undetectable levels of these two cytokines, and patients with chronic HBV without evidence of liver inflammation had no significant increases in IL-8 and IFN-α compared to healthy donors (FIG. 1c,d). Further analysis of these data revealed a similar correlation of raised IL-8 levels and a high HBV viral load (data not shown), supporting the original observation that fluctuations in IL-8 concentrations mirrored those of HBV-DNA (FIG. 1b).

### Direct Ex Vivo Correlation Between NK Cell Expression of the Pro-Apoptotic Ligand TRAIL and HBV-Related Liver Inflammation

A large proportion of IFN-α-activated NK cytotoxicity is mediated through the pro-apoptotic ligand TRAIL (22), recently identified as a major effector in murine models of liver damage (15). Having established that the dominant cytokines during flares were IFN-α and IL-8, the inventors investigated if there was an associated activation of the NK cell TRAIL pathway. Human NK cells have been reported to express little or no TRAIL ligand on their surface when freshly isolated from healthy donor blood (30-32). However, some NK cell TRAIL is detectable upon permeabilisation (30) and they are capable of upregulating it upon activation in culture (31, 32). In contrast, CD3⁻CD56⁺ NK cells from an eAg-CHB patient with recurrent flares were found to have a clear population surface co-staining with an anti-TRAIL mAb directly ex vivo (FIG. 2a). The proportion of NK cells expressing surface TRAIL further increased when ALT was raised (FIG. 2a).

In a subset of five patients from the longitudinal cohort of eAg-CHB patients for whom serial PBMC were available before, during and after flares, the inventors were able to make a temporal analysis of NK cell activation and TRAIL expression. As illustrated for two representative patients in FIG. 2b, surface TRAIL expression on NK cells showed large variations ex vivo concurrent with hepatic flares (see FIG. 2b upper panels). The NK cell expression of CD69, a marker of activation, also correlated tightly with the hepatic flare, with peak activation coinciding with maximal ALT (FIG. 2b upper panels) and with elevated levels of IL-8 and IFN-α (Table 2).

The majority of TRAIL was noted to be on the CD56^{bright} subset of NK cells (see representative sample in FIG. 2a) rather than the larger CD56^{dim} subset responsible for perforin-mediated cytotoxicity (33). The increase in overall NK cell TRAIL expression during flares was noted to be due to an increase in the percent of the CD56^{bright} subset within the NK cells and an increase in the proportion of these CD56^{bright} NK cells expressing TRAIL (FIG. 2b lower panels).

The inventors then compared the level of NK cell surface TRAIL expression in the larger cross-sectional cohort of healthy donors and HBV patients with or without liver inflammation. The percentage of NK cells expressing TRAIL on their surface directly ex vivo was increased more than 4 fold in patients with liver inflammation compared to HBV patients with normal ALT (p<0.001) or healthy donors (p<0.0001)(FIG. 2c). Increased TRAIL expression in patients with liver inflammation compared to patients with no inflammation was also observed within the CD56^{bright} subset (p<0.0005; data not shown).

Of note, levels of TRAIL expressed on CD3⁺ T cells remained low upon longitudinal and cross-sectional analysis of HBV patients, irrespective of the degree of liver inflammation (data not shown). In addition, levels of T cell proliferation to HBV core and surface antigens showed no increase around the time of the flare in the three patients in whom this parameter was examined longitudinally.

These data, showing an in vivo upregulation of activated NK cells expressing TRAIL, suggest a role for NK cells utilising this pathway in the pathogenesis of HBV-induced liver disease.

### Intrahepatic NK Cells Express High Levels of TRAIL and are Highly Activated.

Next the inventors investigated whether the NK cell TRAIL pathway could operate in the liver, the site of active HBV replication. It is already well established that NK cells are enriched in healthy livers compared to the periphery (7). To identify if NK cell numbers are likewise increased in the livers of CHB patients, intrahepatic mononuclear cells were isolated from HBV infected livers (3 with cirrhosis and two with a flare) and the proportions of CD3⁺ T cells, CD3⁻CD56⁺ NK cells and CD3+CD56⁺ NKT cells determined by flow cytometry. As shown in FIG. 3a, both NK and NKT cells were enriched in the liver compared to the periphery of HBV infected patients, with CD3⁻CD56⁺NK cells typically constituting up to 40% of total intrahepatic lymphocytes. This is in line with recent data indicating that NK cells constitute 30-40% of intrahepatic lymphocytes in HBV patients (as in healthy controls), irrespective of viral load, ALT or histology (34).

When the activation status of these intrahepatic NK cells was assessed, a greater proportion of intrahepatic NK cells had upregulated CD69 than peripheral NK cells from the same patient (FIG. 3b). Of note, the most highly activated NK cell subset in the liver was the CD56^{bright} subset (FIG. 3c), a subset that was also preferentially enriched in the liver (data not shown).

A recent paper by Ishiyama et al showed that TRAIL was not detectable on NK cells extracted from healthy livers at the time of living donor transplantation (32). By contrast the inventors found that intrahepatic NK cells isolated from these HBV-infected livers expressed TRAIL directly ex vivo, at even higher levels than seen in the periphery of the same patients (FIG. 3d). As in the periphery, TRAIL was predominantly expressed on the preferentially activated CD56^{bright} NK subset, and intrahepatic CD3⁺ T cells expressed little TRAIL (FIG. 3e).

To further examine the relationship between intrahepatic NK TRAIL levels and liver inflammation, the inventors compared five biopsies taken around the time of an ALT flare in patients with histologically proven eAg-CHB with two biopsies from HBV patients with normal ALT and histology confirming inactive disease. TRAIL-positive lymphocytes (presumed to be NK cells since these are the only population expressing significant TRAIL in the periphery or liver) were identified in 4 out of the 5 eAg-CHB sections by immunohistochemistry (FIG. 3f). By contrast, sections from the two patients with inactive HBV infection resembled reports of healthy livers (32), with no TRAIL-expressing lymphocytes identifiable. The results suggest that intrahepatic NK cell TRAIL expression correlates with HBV-related liver inflammation.

NK Cells from Patients with Chronic HBV Infection can be Activated and Induced to Express TRAIL by Cytokine Concentrations Found During Liver Inflammation

The inventors next sought to explore possible mechanistic links between the ex vivo findings of increases in IFN-α, IL-8 and NK-expressed TRAIL in patients with raised ALT. These two cytokines found in high concentrations during HBV-related inflammation could contribute to liver damage by immunomodulatory effects on NK cells and the TRAIL pathway. IFN-α is a modulator of NK cell function but it was unclear how this would be affected by IL-8, an inhibitor of its antiviral efficacy (26, 27). Furthermore, it was possible that NK cells could become resistant to IFN-α-mediated modulation after the recurrent stimulation likely in these patients with longstanding HBV-related inflammation. To investigate this, PBMC or purified NK cells from healthy volunteers and patients with chronic HBV were incubated in vitro for 24 hours with IFN-α or IL8 alone or in combination, at concentrations observed during hepatic flares. PBMC or purified NK cells showed a substantial increase in the percentage of NK cells expressing TRAIL upon incubation with IFN-α (FIG. 4a). IL-8 did not have a direct effect or inhibit the ability of IFN-α to upregulate TRAIL expression. Rather than becoming resistant to the effects of IFN-α, NK cells from chronically infected HBV patients, including patients undergoing flares, upregulated TRAIL by a similar amount to NK cells from healthy donors (FIG. 4a). HBV patients with liver inflammation therefore achieved a higher total NK cell TRAIL level after in vitro IFN-α treatment as a result of their higher starting expression ex vivo. NK cells taken from patients with chronic HBV infection also maintained the capacity to be activated by IFN-α, with equivalent levels of CD69 upregulation to that seen in NK cells from healthy donors and again no inhibition of this effect by IL-8 (FIG. 4b). IFN-α induced equivalent levels of activation of highly purified NK cells, indicating a direct effect of this cytokine (data not shown). Thus the in vivo observations of upregulation of NK cell TRAIL and CD69 expression were mirrored in vitro using equivalent concentrations of cytokines to those circulating in CHB patients with liver inflammation.

### Cytokine-Modulated TRAIL Receptor Expression on Hepatocytes in HBV Infection

In order for TRAIL to induce receptor mediated cell death, it needs to engage with a death domain receptor on the target cell (14). Previous studies have suggested minimal protein expression of TRAIL death-inducing receptors in healthy livers (19, 35). However, mRNA for the death-inducing receptors TRAIL-R1 and TRAIL-R2 has been isolated from human hepatocytes (14, 17), which become susceptible to TRAIL-induced apoptosis upon culture (17), suggesting a potential for upregulation. To ascertain whether hepatocytes in HBV-infected livers express a death-inducing receptor that could engage with the NK-expressed TRAIL, paraffin-embedded liver sections from HBV-infected and control livers were stained for TRAIL-R1 and TRAIL-R2. No TRAIL-R1 was detected in the HBV-infected or control livers (data not shown). However, TRAIL-R2 was expressed by hepatocytes in ten of the thirteen HBV-infected liver sections stained (from patients with eAg-CHB, with histology showing mild to moderate inflammatory infiltrates or cirrhosis). Immunostaining for TRAIL-R2 was predominantly localised to the surface of hepatocytes (FIG. 5a, ×1000 magnification), and ranged from strong in 3 patients, moderate in 2 and weak in 6, with no clear correlation with stage of liver disease. However TRAIL-R2 was absent in sections from two control HBV patients with normal ALT and inactive disease. TRAIL-R2 staining was detected in a control donor with hepatic steatosis, but not in the other control liver sections examined, 3 from healthy donors, 4 from patients with alcoholic hepatitis (FIG. 5b).

The inventors hypothesised that IFN-α or IL-8 might partially mediate this altered TRAIL-R expression pattern observed in HBV-infected inflamed livers to favour hepatocyte death. In order to investigate this, HepG2 hepatocytes were incubated with IL-8 or IFN-α at equivalent concentrations to those circulating in patients, and the levels of expression of the death-inducing (TRAIL-R1 and -R2) and inhibitory (TRAIL-R3 and -R4) TRAIL receptors determined by flow cytometry. IL-8 was consistently found to induce an approximate doubling in the expression of TRAIL-R2 (FIG. 5c), the death domain receptor observed to be upregulated on hepatocytes of CHB patients (FIG. 5a). Incubation with IL-8 did not alter surface expression of any of the other TRAIL-R (data not shown). IFN-α had no effect on the expression of TRAIL-R2 (data not shown), but reproducibly and substantially decreased expression of the decoy/regulatory receptor TRAIL-R4 (FIG. 5d). TRAIL-R4 has recently been shown to form a ligand-independent association with TRAIL-R2 to inhibit apoptosis induction (36). Taken together, these results suggest that the high concentrations of IL-8 and IFN-α can act in combination to both increase a death-inducing receptor and reduce an inhibitory receptor, thus optimally predisposing hepatocytes to TRAIL mediated cell death.

### Cytokines Circulating During HBV Flares can Render NK Cells Capable of Killing Hepatocytes Through Trail Ligand/Receptor Interactions

To test whether NK cells isolated from HBV patients could kill hepatocytes using TRAIL the inventors utilised an assay that can directly measure the degree of receptor mediated cell death via the caspase cascade pathway utilised by TRAIL. PBMC from patients were incubated with or without IFN-α overnight to induce maximal TRAIL expression on the NK cells. At the same time, HepG2 hepatoma cells were pre-incubated with or without IL8 overnight. Activated PBMC were then added to the HepG2 cells and the degree of HepG2 cell caspase activation assessed by flow cytometry. When the HepG2 cells or PBMC were not treated with cytokines there was little caspase activation when compared to the background HepG2 cell levels (data not shown). Pre-treatment of the HepG2 cells with IL-8 increased the amount of PBMC-mediated caspase activation, which was further increased when the PBMC were pre-incubated with IFN-α (FIG. 6a upper panel).

In order to confirm that this IFN-α induced caspase activation was TRAIL-mediated, the HepG2 cells were pre-incubated with a blocking antibody to TRAIL. As can be seen in FIG. 6a lower panel, when TRAIL was blocked there was a reduction in IFN-a induced caspase activation compared to the non-antibody treated cells. This suggests that TRAIL plays a major role in the IFN-α induced caspase activation but may not be the only mechanism involved. The IFN-α-induced increase in TRAIL-mediated death was maintained using purified NK cells, and abrogated in NK cell depleted fractions (data not shown).

PBMC from HBV patients without liver inflammation or from healthy controls showed less efficient initiation of the caspase cascade following up-regulation of NK cell TRAIL with in vitro IFN-α treatment (FIG. 6b). There was twice as much caspase induction using IFN-α-activated PBMC from flaring patients (FIGS. 6a and b) as from healthy donors (FIG. 6b). PBMC taken from patients with HBV-related liver inflammation were also able to induce apoptosis when added to HepG2 directly ex vivo, partially blocked in all cases upon addition of a TRAIL-blocking mAb (FIG. 6c). Patients with HBV liver inflammation (n=6), showed a mean of 28% caspase induction over background, which was significantly greater than that seen using PBMC from HBV patients with normal ALT or healthy controls (n=6) (FIG. 6 d,e).

These experiments therefore confirm that, under the influence of cytokines induced during HBV flares, NK cells become capable of inducing death of HepG2 hepatoma cells through the TRAIL pathway.

The experiment described above was repeated but instead of adding in IL-8, the inventors blocked endogenous IL-8 with an anti-IL-8 blocking mAb. NK cells were activated to express TRAIL using IFN-α and apoptosis measured using the FLICA assay for caspase activation, as previously. FIGS. 8a and 8b show the results obtained using PBMC cells from for 2 donors. In both cases anti-IL-8 blocked endogenous IL-8 and resulted in a reduction in apoptosis of hepatocytes. In one case there was an additive effect of anti-IL-8 and anti-TRAIL and in the other there was more marked inhibition of hepatocyte death with the anti-IL8 than with the anti-TRAIL. In both cases the combination of the anti-IL-8 and anti-TRAIL was superior to the use of anti-TRAIL alone. As the levels of IL-8 will be considerably higher in vivo than in vitro, the effect of blocking IL-8 or IL-8 and TRAIL with be more significant in vivo. Furthermore since IL-8 is principally produced by hepatocytes, blocking IL-8 may additionally prevent the chemotaxis effects of IL-8 in attracting NK cells.

NK Cells from HBV Patients with Flares can Initiate TRAIL-Induced Apoptosis of Primary Human Hepatocytes.

The HepG2 hepatoma cell line provided a convenient model to dissect the mechanisms of activation of this pathway, but it was important to confirm that primary human hepatocytes would also be susceptible to NK TRAIL-mediated apoptosis. Hepatocytes were isolated by perfusion of a non-diseased liver explant and cultured for 48 hours, with IFN-α and IL-8 added for the last 24 hours to modulate TRAIL-receptor expression. Viability of hepatocytes without the addition of PBMC was good (greater than 80% in all wells) (FIG. 7a). By contrast, hepatocytes incubated with PBMC from an HBV patient with a flare who had TRAIL-expressing NK cells directly ex vivo, showed hepatocyte apoptosis induction (FIG. 7b). IFN-α-treated PBMC taken from patients expressing NK cell TRAIL during an episode of liver inflammation were more efficient at induction of apoptosis of primary human hepatocytes than PBMC from HBV patients without a flare or healthy donors (p=0.02, MannWhitney U test, FIG. 7c). In 3 out of 4 high ALT patients, more than 30% of the apoptosis induced by IFN-α-treated PBMC could be blocked through TRAIL (mean of 28% blocking for the 4 patients). Induction of apoptosis by PBMC cultured without IFN-α for 24 hours was less reliably elicited (showing a mean 15% increase over background levels in patients with liver disease p=0.04, and a non-significant trend to increased levels in this group compared to controls, FIG. 7c); a larger study using PBMC directly ex vivo will be required to confirm differences between patient groups. However the inventors can conclude that PBMC from HBV patients with liver inflammation whose NK cells express TRAIL are capable of mediating death of primary human hepatocytes.

### The Role of IL-8 in Chemotaxis of NK Cells to the HBV Liver

The inventors have already identified IL-8 to be elevated in the circulation of patients with high level HBV infection compared to low level carriers or healthy donors (see above). The inventors have now shown that the HBV-infected liver is a source of this IL-8. The inventors did this by staining eleven sections of human liver obtained from liver explants and biopsies from patients with HBV-related flares or cirrhosis with a monoclonal specific for IL-8 (R&D Systems), detected using the Dako Chemate Envision horseradish peroxidase kit. Immunohistochemistry revealed strong IL-8 staining in all HBV livers, compared to little or no staining in eight control liver sections from patients with other liver diseases including alcoholic hepatitis (representative staining in FIG. 9).

The inventors have investigated whether NK cells from patients with HBV infection express the high affinity receptor (CXCR1) for IL-8, which should allow them to respond to the IL-8 signals. The inventors stained PBMC directly ex vivo from patients with low or high level HBV infection versus healthy controls with a monoclonal to CXCR1 and identified the NK cells as CD3 negative and CD56 positive (monoclonals from R&D Systems). The inventors found high levels of expression of CXCR1 on NK cells from HBV patients, including on the CD56^{bright} subset known to express TRAIL (FIG. 10).

The inventors have also demonstrated that these receptors were functional by showing migration of the NK cells from HBV patients towards the IL-8 ligand in vitro. This was done using a transwell system (ChemoTX from Neuroprobe) and using concentrations of recombinant IL-8 (R&D Systems) found in HBV patients. PBMC were added to the upper chamber, recombinant IL-8 (concentrations between 5 and 500 ng/ml) to the lower chamber and after 2 hours incubation at 37° C., the composition of the migrated cells compared to that with no chemokine by flow cytometry (using NK cell-specific monoclonals described above).

The inventors then developed an assay to show that these migrated NK cells were capable of killing human hepatocytes. The inventors modified the transwell system above by adding the HepG2 human hepatoma cell line (10,000 cells per chamber, which had been optimised for TRAIL receptor expression as described previously) to the bottom well. The inventors showed that upon addition of IL-8 to the bottom chamber and PBMC to the top chamber, it was possible to induce migration of NK cells that were capable of killing the HepG2 cells. This killing was measured using the FLICA assay for caspase activation described previously. In FIG. 11 representative increases in FLICA (hepatocyte killing) upon migration of PBMC induced with 500 ng/ml of recombinant IL-8 compared to background with media alone are shown.

### Discussion

The protracted, unpredictable natural history of the development of liver disease in chronic HBV infection makes it difficult to sample the immune correlates of liver damage longitudinally. Recurrent hepatic flares occurring on a background of chronic HBV overcome this problem by allowing capture of a compressed version of immunopathogenetic events associated with rapid changes in liver disease and viral load. Previous studies have examined the flares associated with eAg to antiHBe seroconversion and those found in patients undergoing therapy, demonstrating increases in serum IL-12 (37) and CD4 T cell reactivity (37-39). In this study the inventors focused initially on the distinct type of flare seen in patients with late reactivation of their disease, so called eAg-CHB. These patients usually have mutations in their basal core promoter region or stop codon resulting in loss of eAg expression in the face of high viral load, and are at particularly high risk of progression to fibrosis and cirrhosis (1, 2). By repeatedly sampling a cohort of patients with eAg-CHB, the inventors were able to identify raised and highly fluctuating levels of IL-8 and IFN-α during flares. The proportion of NK cells activated to express CD69 and the apoptosis-inducing TRAIL ligand directly ex vivo also fluctuated in parallel with the hepatic flares. A larger cross-sectional study extended the finding of elevated levels of serum IL-8, IFN-α and NK cell TRAIL to patients with HBV infection with active liver inflammation as opposed to healthy HBV carriers or controls. TRAIL-expressing NK cells were further enriched and activated in the liver of HBV patients, contrasting with the lack of intrahepatic TRAIL expression ex vivo in healthy controls (32). Investigation of the possible mechanistic links between the induction of these cytokines and of the NK cell TRAIL pathway revealed that IL-8 is capable of up-regulating a death-inducing receptor for TRAIL, increased expression of which was observed in CHB livers. IFN-α, at concentrations circulating during flares, could promote cell death through the TRAIL pathway both by inducing ligand expression on NK cells and by reducing inhibition by a regulatory receptor on hepatocytes. Together, they render NK cells capable of killing hepatocytes through TRAIL.

NK cells are highly enriched in the liver of both healthy donors and HBV patients, comprising the dominant intrahepatic lymphocyte population, yet their role in HBV-related liver damage has not been well defined. Here the inventors present data supporting an important contribution of NK cells to HBV-related liver damage, showing activation of NK cells in parallel with flares of liver inflammation and enrichment of activated NK cells in the HBV-infected liver. The CD56^{dim} subset expresses the majority of NK cell perforin and granzyme, but hepatocytes are relatively resistant to these classical cytolytic effector molecules (9, 10). The CD56^{bright} subset of NK cells, noted to be selectively enriched in the periphery during flares and preferentially enriched and activated in the liver, is known for its immunoregulatory capacity, being a potent source of cytokines such as IFN-γ (33). In this study the inventors have concentrated on the potential of these CD56^{bright} NK cells to mediate liver damage through an alternative cytotoxic pathway, utilising TRAIL to induce receptor-mediated hepatocyte death. TRAIL has been shown to be endogenously expressed by a subset of NK cells found in murine livers (16) but this is not the case in humans, where both peripheral and intrahepatic NK cells show minimal surface TRAIL expression in healthy individuals (30-32). However human NK cells have been reported to be capable of upregulating TRAIL expression upon stimulation in vitro with IL-2 (31, 32) or IFN-α (22); the inventors demonstrate that NK cells retain the capacity to upregulate TRAIL both in vitro and in vivo, despite the years of recurrent inflammation seen in these patients with chronic HBV infection. The fact that NK cell TRAIL is only elevated in those HBV patients manifesting liver inflammation (in both longitudinal and cross-sectional studies) supports a role for this ligand in hepatocyte damage.

The TRAIL pathway was originally proposed to be restricted to transformed cells, and NK-expressed TRAIL protects against tumours in the intrahepatic environment (16). However, recent human studies have highlighted a pathogenic role for this pathway outside the context of tumours, with lymphocytes mediating TRAIL-induced apoptosis of atherosclerotic plaques in acute coronary syndrome (40), and of CD4 T cells in HIV infection (41). Studies in mouse models of liver disease have reinforced the notion of NK-expressed TRAIL inducing damage of non-malignant tissues in vivo, showing TRAIL-dependent death of hepatocytes (15) and hepatic stellate cells (42). The susceptibility of human hepatocytes to TRAIL-induced apoptosis has been an area of controversy, following initial reports of lack of liver toxicity in mice and primates treated with soluble TRAIL (43, 44). However membrane-bound TNF-related ligands have greater pro-apoptotic potential and liver toxicity than their soluble counterparts (35, 45). Human membrane-bound TRAIL does induce hepatocyte apoptosis in mice, resulting in widespread apoptosis, necrosis and lymphocytic infiltration (35), compatible with the pathology of chronic HBV hepatitis. Furthermore, normal human hepatocytes have the potential to express death-inducing receptors for TRAIL and are susceptible to TRAIL-induced apoptosis in vitro (17, 18). The ratio of expression of death-inducing versus regulatory receptors has been shown to provide a means for fine-tuning the susceptibility to TRAIL-induced death (36). There is already a suggestion that this balance may be tipped in favour of death in situations of liver inflammation such as bile acid retention (46) and viral hepatitis. Evidence for the latter comes from immunostaining of hepatitis C virus-infected livers (20) and western blotting of total liver extracts from acute HBV-mediated liver failure (19). The inventors show by immunostaining that expression of a death-inducing TRAIL receptor is upregulated on hepatocytes of patients with CHB. One mechanism of modulation may be by the virus itself, based on the in vitro observations that the HBV-encoded X antigen upregulates one of the death-inducing receptors (47) and predisposes to TRAIL-induced apoptosis through modulation of intracellular Bax (48). Here the inventors demonstrate an additional mechanism, whereby cytokines produced during an HBV flare may act in concert to both increase death-inducing, and reduce regulatory TRAIL receptors in order to maximise hepatocyte apoptosis. The data supports the use of soluble TRAIL in the therapy of malignancies such as hepatocellular carcinoma. They suggest that tumour patients with coincident HBV infection and episodes of active liver inflammation might be more susceptible to hepatic toxicity from such a therapeutic approach.

The chemokine ligand/receptor pairs directing the migration of this large influx of NK cells into the HBV-infected liver have been dissected. As indicated above, IL-8 induces chemotaxis of NK cells at doses equivalent to those found in HBV patients. IL-8 is well-known for its chemotactic function and the high concentrations circulating during flares are likely to derive from the liver. In the patients studied here, IL-8 levels typically increased with the increase in HBV DNA, in keeping with the reported ability of HBV to transactivate the IL-8 gene (49). NK cells have been shown to express the high affinity IL-8 receptor CXCR1, and to migrate in response to IL-8 (50). Interferons have also been shown to regulate the trafficking of NK cells to the liver by induction of chemokines such as interferon-gamma inducible protein (IP-10) in HBV transgenic mice (4) and MIP-1α in murine CMV infection (51). The inventors have not shown where the IFN-α surges identified in this study derive from, but likely sources are virally infected hepatocyes in addition to liver-infiltrating leukocytes including plasmacytoid dendritic cells. The inventors have demonstrated that IL-8 and possibly IFN-α, in addition to activating a pathway of NK-mediated hepatocyte damage, contribute to the chemotaxis of NK cells to the HBV-liver during episodes of active inflammation.

In the transgenic mouse model of HBV infection, NK cells have potent antiviral efficacy, an effect that is attenuated in mice lacking the Type I interferon receptor (52). It is likely that IFN-α-activated NK cells have a dual role in viral control and liver damage in human HBV infection too. TRAIL-induced apoptosis of HBV-infected hepatocytes by NK cells would eliminate some virally infected cells, a process that could contribute to the partial reduction in viral load often observed after a flare. However, any viral reduction by this means would always be at the expense of liver damage and would therefore be a hazardous strategy to promote therapeutically. In fact, the use of exogenous IFN-α in the treatment of HBV-associated cirrhosis is often limited by its tendency to cause a hepatic flare, which can be severe enough to precipitate hepatic decompensation. As indicated herein, by blocking IL-8 and for the TRAIL pathway it is possible to limit hepatocyte apoptosis associated with liver disease and/or IFN-α therapy.

All documents cited are incorporated herein by reference.

TABLE 1
IL-8 and IFN-α levels are highly elevated in sera from eAg-CHB
patients with flares. The median value is that of the maximum cytokine
concentration obtained from 12 CHB patients undergoing flares of
liver inflammation and 14 healthy control donors.

Median of peak
cytokine conc. (pg/mL)

| | CHB patients | Healthy Controls | p-values |
|---|---|---|---|
| IL-8 | 630 | 13 | <0.0001 |
| IL-1β | 79 | blq | 0.07 |
| IL-6 | 11 | blq | 0.08 |
| IL-10 | 9 | 1.7 | 0.04 |
| TNF | 4 | blq | 0.64 |
| IL-12p70 | 17 | 2.6 | 0.1 |
| IFN-α | 253 | 20 | 0.005 |

blq = below level of quantification.
Significance testing was done using the Mann Whitney U test, with those of statistical significance highlighted in bold.

TABLE 2
Large fold increases in IL-8 and IFN-α serum concentrations
during hepatic flares.

| | | | IL-8 | | IFN-α | |
|---|---|---|---|---|---|---|
| | | | | No. of | | No. of |
| | | | | samples | | samples |
| | | Max | Max | from | Max | from |
| | No. of | ALT | fold | peak ALT | fold | peak ALT |
| | samples | (IU/L) | change^{a} | level | change | level |
| Patient 1 | 7 | 546 | 136 | −1 | 152 | −3 |
| Patient 2 | 10 | 285 | 686 | 0 | 7 | 0 |
| Patient 3 | 7 | 569 | 237 | −2 | 6.8 | −1 |
| Patient 4 | 8 | 208 | 417 | −2 | 12 | na |
| Patient 5 | 8 | 495 | 40 | 2 | 7.2 | −5 |
| Patient 6 | 7 | 313 | 8.2 | −1 | 7 | 0 |
| Patient 7 | 7 | 214 | 29 | −1 | 20 | 0 |
| Patient 8 | 5 | 201 | 303 | −3 | 3.7 | 0 |
| Patient 9 | 4 | 565 | 11 | 0 | 2.4 | −1 |
| Patient | 4 | 880 | 1.7 | −3 | 3.2 | 0 |
| 10 | | | | | | |
| Patient | 5 | 403 | 5.2 | 0 | 2.3 | 2 |
| 11 | | | | | | |
| Patient | 5 | 614 | 5.4 | −1 | 1.4 | −2 |
| 12 | | | | | | |
| Patient | 5 | 158 | 32 | 0 | 8.2 | 1 |
| 13 | | | | | | |
| Patient | 4 | 196 | 70 | −1 | 9.5 | 0 |
| 14 | | | | | | |
| Median | | | 36 | −1 | 7 | 0 |

^{a}Max Fold Change is the fold change of IL-8 or IFN-α from baseline levels to the peak of the cytokine fluctuation.
na—data not available.

### REFERENCES

1. Bonino, F., and M. R. Brunetto et al., J Hepatol 39 Suppl, 1, S160-163, 2003.
2. Brunetto, M. R. et al., J Hepatol, 36, 263-270, 2002.
3. Maini, M. K. et al., J Exp Med, 191, 1269-1280, 2000.
4. Kakimi, K. et al., J Exp Med. 194, 1755-1766, 2001.
5. Sitia, G. et al., J Clin Invest, 113, 1158-1167, 2004.
6. Ando, K. et al., J Exp Med, 178, 1541-1554, 1993.
7. Norris, S et al., J Hepatol, 28, 84-90, 1998.
8. Webster, G. J. et al., Hepatology, 32, 1117-1124, 2000.
9. Tay, C. H., and R. M. Welsh. J et al., Virol, 71, 267-275, 1997.
10. Kafrouni, M. I. et al., J Immunol, 167, 1566-1574, 2001.
11. Galle, P. R. et al., J Exp Med. 182, 1223-1230, 1995.
12. Balkow, S. et al., J Virol, 75, 8781-8791, 2001.
13. Wiley, S. R. et al., Immunity, 3, 673-682, 1995.
14. Sheridan, J. P. et al., Science, 277, 818-821, 1997.
15. Zheng, S. J. et al., J Clin Invest, 113, 58-64, 2004.
16. Takeda, K. et al., Nat Med. 7, 94-100, 2001.
17. Jo, M. et al., Nat Med, 6, 564-567, 2000.
18. Lawrence, D. et al., Nat Med. 7, 383-385, 2001.
19. Mundt, B. et al., Faseb J, 17, 94-96, 2003.
20. Saitou, Y. et al., Hum Pathol, 36, 1066-1073, 2005.
21. Nguyen, K. B. et al., J Immunol, 169, 4279-4287, 2002.
22. Sato, K. et al., Eur J Immunol, 31, 3138-3146, 2001.
23. Marrack, P., J. Kappler, and T. Mitchell et al., J Exp Med, 189, 521-530, 1999.
24. Dondi, E. et al., J Immunol, 170, 749-756, 2003.
25. Tian, Z. et al., X. J Immunol, 165, 3959-3965, 2000.
26. Khabar, K. S. et al., J Exp Med, 186, 1077-1085, 1997.
27. Polyak, S. J. et al., J Virol, 75, 6209-6211, 2001.
28. Guidotti, L. G. et al., Science 284, 825-829, 1999.
29. Myers, R. P. et al., J Hepatol, 39, 222-230, 2003.
30. Kashii, Y. et al., J Immunol, 163, 5358-5366, 1999.
31. Mirandola, P. et al., Blood, 104, 2418-2424, 2004.
32. Ishiyama, K. et al., Hepatology, 43, 362-372, 2006.
33. Cooper, M. A. et al., Trends Immunol, 22, 633-640, 2001.
34. Sprengers, D. et al., J Med Virol, 78, 561-568, 2006.
35. Ichikawa, K. et al., Nat Med. 7, 954-960, 2001.
36. Clancy, L. et al., Proc Natl Acad Sci USA, 102, 18099-18104, 2005.
37. Rossol, S. et al., J Clin Invest, 99, 3025-3033, 1997.
38. Tsai, S. L. et al., J Clin Invest, 89, 87-96, 1992.
39. Lohr, H. F. et al., Hepatology, 22, 61-68, 1995.
40. Sato, K. et al., J Exp Me, 203, 239-250, 2006.
41. Herbeuval, J. P. et al., Blood, 106, 3524-3531, 2005.
42. Radaeva, S. et al., Gastroenterology, 130, 435-452, 2006.
43. Ashkenazi, A. et al., J Clin Invest, 104, 55-162, 1999.
44. Walczak, H. et al., Nat Med. 5, 157-163, 1999.
45. Schneider, P. et al., J Exp Med, 187, 1205-1213, 1998.
46. Higuchi, H. et al., J Biol Chem, 276, 38610-38618, 2001.
47. Janssen, H. L. et al., J Hepatol, 39, 414-420, 2003.
48. Liang, X. et al., J Immunol, 178, 503-510, 2007.
49. Mahe, Y. et al., J Biol Chem, 266, 13759-13763, 1991.
50. Campbell, J. J. et al., J Immunol, 166, 6477-6482, 2001.
51. Salazar-Mather, T. P. et al., J Clin Invest, 110, 321-330, 2002.
52. Kakimi, K. et al., J Exp Med. 192, 921-930, 2000.
53. Kayagaki, N. et al., J Exp Med, 189, 1451-1460, 1999.
54. Reynolds, G. M. et al., Br J Haematol, 118, 195-201, 2002.
55. Afford, S. C. et al., J Exp Med, 189, 441-446, 1999.
56. Liu, Yu-Gang et al., Biochemical and Biophysical Research Communications, 352, 329-334, 2007.

1. (canceled)

2. (canceled)

3. (canceled)

4. (canceled)

5. The method of claim 29 further comprising delivering an effective amount of an IL-8 blocking agent to the individual.

6. (canceled)

7. (canceled)

8. (canceled)

9. (canceled)

10. The method of claim 26 or 30, wherein the IL-8 blocking agent blocks the activity of IL-8 to induce the expression of TRAIL-R2 on hepatocytes.

11. The method of claim 10, wherein the IL-8 blocking agent is selected from the group consisting of soluble IL-8 receptors and antibody molecules having affinity for IL-8.

12. The method of claim 29 or 33, wherein the TRAIL blocking agent prevents the interaction between a TRAIL ligand and a TRAIL death inducing receptor.

13. The method of claim 12, wherein the TRAIL blocking agent is selected from the group consisting of soluble TRAIL receptors, antibody molecules having affinity for the TRAIL ligand or a TRAIL death inducing receptor.

14. The method of claim 26, 29, or 30, wherein the liver disease is associated with HBV infection in the individual.

15. The method of claim 14, wherein the liver disease is a chronic HBV infection.

16. The method of claim 14, wherein the liver disease is an eAg-CHB infection.

17. The method of claim 14, wherein the liver disease exhibits hepatic flares.

18. The method of claim 26, 29, 30, or 33, further comprising delivering an effective amount of IFN-α or an antiviral reverse transcriptase inhibitor, or an nucleic acid encoding IFN-α or an antiviral reverse transcriptase inhibitor to the individual.

19. (canceled)

20. The method of claim 26, 29, 30, or 33 further comprising delivering an effective amount of a HBV antiviral agent to the individual.

21. (canceled)

22. The method of any on of claim 26, 29, 30, or 33 wherein the individual is a human.

23. (canceled)

24. An expression vector encoding a TRAIL blocking agent and an IL-8 blocking agent.

25. A host cell transformed with one or more nucleic acid molecules encoding a TRAIL blocking agent and an IL-8 blocking agent.

26. A method for the treatment and/or prophylaxis of an individual with liver disease comprising delivering an effective amount of an IL-8 blocking agent to the individual.

27. The method of claim 26, wherein a TRAIL blocking agent is not administered to the individual.

28. The method of claim 26, which additionally comprises delivering a TRAIL blocking agent to the individual.

29. A method for the treatment and/or prophylaxis of an individual with hepatic flares comprising delivering an effective amount of a TRAIL blocking agent to the individual.

30. A method for the treatment and/or prophylaxis of an individual with liver disease comprising delivering an effective amount of one or more nucleic acid molecules encoding an IL-8 blocking agent to the individual.

31. The method of claim 30, a TRAIL blocking agent or an nucleic acid encoding a TRAIL blocking agent is not delivered to the individual.

32. The method of claim 30, which additionally comprises delivering a TRAIL blocking agent or a nucleic acid encoding a TRAIL blocking agent to the individual.

33. A method for the treatment and/or prophylaxis of an individual with hepatic flares comprising delivering an effective amount of one or more nucleic acid molecules encoding a TRAIL blocking agent to the individual.

34. The method of any one of claims 26, 30, or 33 additionally comprising delivering an effective amount of IFN-α or an antiviral reverse transcriptase inhibitor, or a nucleic acid encoding IFN-α or an antiviral reverse transcriptase inhibitor, to the individual.

35. A pharmaceutically acceptable composition comprising a TRAIL blocking agent and an IL-8 blocking agent, or one or more nucleic acids encoding a TRAIL blocking agent and an IL-8 blocking agent, together with one or more pharmaceutically acceptable excipients.

36. The composition of claim 35 which additionally comprise IFN-α or an antiviral reverse transcriptase inhibitor, or a nucleic acid molecule encoding IFN-α or an antiviral reverse transcriptase inhibitor.

37. A pharmaceutically acceptable composition comprising a TRAIL blocking agent in combination with IFN-α or a reverse transcriptase antiviral, or one or more nucleic acid molecules encoding a TRAIL blocking agent and IFN-α or a reverse transcriptase antiviral, together with one or more pharmaceutically acceptable excipients.

38. A pharmaceutically acceptable composition comprising an IL-8 blocking agent in combination with IFN-α or a reverse transcriptase antiviral, or one or more nucleic acid molecules encoding an IL-8 blocking agent and IFN-α or a reverse transcriptase antiviral, together with one or more pharmaceutically acceptable excipients.

39. A kit for treating a liver disease comprising a TRAIL blocking agent and an IL-8 blocking agent, or one or more nucleic acids encoding a TRAIL blocking agent and an IL-8 blocking agent.

40. The kit of claim 39 which additionally comprising IFN-α or a reverse transcriptase antiviral, or a nucleic acid molecule encoding IFN-α or a reverse transcriptase antiviral.

41. A kit for treating hepatic flares comprising a TRAIL blocking agent in combination with IFN-α or a reverse transcriptase antiviral, or one or more nucleic acid molecules encoding a TRAIL blocking agent and IFN-α or a reverse transcriptase antiviral.

42. A kit for treating a liver disease comprising an IL-8 blocking agent in combination with IFN-α or a reverse transcriptase antiviral, or one or more nucleic acid molecules encoding an IL-8 blocking agent and IFN-α or a reverse transcriptase antiviral.

43. (canceled)

44. (canceled)

45. (canceled)

46. (canceled)
